(19) United States
(12) Patent Application Publication
(10) Pub. No.: US 2025/0325358 A1
(43) Pub. Date: Oct. 23, 2025

(54) SEMI-AUTOMATIC ADJUSTABLE-DOSE SYRINGE

(71) Applicant: Hui Zhou, Tongliao (CN)

(72) Inventor: Hui Zhou, Tongliao (CN)

(21) Appl. No.: 19/182,275

(22) Filed: Apr. 17, 2025

(30) Foreign Application Priority Data
Apr. 17, 2024 (CN) 202410464056.6

(51) Int. Cl. A61D 1/02

(52) U.S. Cl. CPC A61D 1/025

(57) ABSTRACT

Disclosed is a semi-automatic adjustable-dose syringe, including a syringe cylinder, an adjustment cylinder, a piston sleeve rod, a T-shaped piston adjustment rod, a first spring, an outer pull cap, second springs and a needle break-proof buffer cap. An exterior of a left side of the syringe cylinder is threadedly connected to the adjustment cylinder through mutual engagement; and the adjustment cylinder is internally housed within the outer pull cap, with a sliding connection between an outer wall of the adjustment cylinder and an inner wall of the outer pull cap. The present disclosure can prolong the service life of the syringe by changing and adjusting structures and component combinations of the syringe itself, which effectively reduces wear and waste of components, ensures safe operation and saves time and labor, significantly minimizing physical harm and energy consumption of epidemic prevention personnel.

### CROSS-REFERENCE TO RELATED APPLICATION

This application claims priority of Chinese Patent Application No. 202410464056.6, filed on Apr. 17, 2024, the entire contents of which are incorporated herein by reference.

### TECHNICAL FIELD

The present disclosure relates to the technical field of an auxiliary device for breeding industry, and specifically to a semi-automatic adjustable-dose syringe.

### BACKGROUND

In livestock farms, vaccination is the most effective prevention and control measure. However, during the vaccination process for livestock and poultry, stress reactions of animals still pose potential hazards that cannot be overlooked, both to the animals themselves and to medical personnel. Furthermore, existing syringes currently in use suffer from rapid component wear and tear, resulting in a short service life.

Therefore, the present disclosure provides a semi-automatic adjustable-dose syringe, which prolongs service life by changing and adjusting structures and component combinations of the syringe itself, effectively reducing wear and waste of components, ensuring safe operation, and fully demonstrating more reasonable ergonomic design methodology.

### SUMMARY

In response to the shortcomings of the above related art, the present disclosure aims to provide a semi-automatic adjustable-dose syringe.

To solve the above technical problems, the present disclosure employs the following technical solutions.

The present disclosure provides a semi-automatic adjustable-dose syringe, including a syringe cylinder, an adjustment cylinder, a piston sleeve rod, a T-shaped piston adjustment rod, a first spring, an outer pull cap, second springs and a needle break-proof buffer cap. An exterior of a left side of the syringe cylinder is threadedly connected to the adjustment cylinder through mutual engagement; the adjustment cylinder is internally housed within the outer pull cap, with a sliding connection between an outer wall of the adjustment cylinder and an inner wall of the outer pull cap; the piston sleeve rod is internally housed within the outer pull cap, one end of the piston sleeve rod is clamped into a clamping groove in the outer pull cap through mutual engagement, and the other end of the piston sleeve rod penetrates through a through hole at a left end of the adjustment cylinder and extends into the syringe cylinder; and the needle break-proof buffer cap is sleeved on a left end of the outer pull cap, and an outer side face of the left end of the outer pull cap is movably connected to an inner side face of a left end of the needle break-proof buffer cap through the second springs.

The first spring is sleeved on an outer side of the clamping groove inside the outer pull cap, one end of the first spring is adhered to the left end of the interior of the outer pull cap, and the other end of the first spring penetrates through the through hole on the adjustment cylinder and is adhered to an outer side face of a left end of the syringe cylinder; a connector matched with a mounting cap in a steel needle through a snap-fit connection is arranged at the left end of the outer pull cap; and the T-shaped piston adjustment rod is internally housed within the syringe cylinder, and a hollow rod in the T-shaped piston adjustment rod is inserted into the piston sleeve rod and is threadedly connected to internal threads on an inner side of the connector through mutual engagement.

Preferably, the syringe cylinder is formed by a transparent inner cylinder and a metal outer cylinder from inside to outside, with the metal outer cylinder sleeved on the transparent inner cylinder; external threads matched with inner threads in the adjustment cylinder are disposed on the metal outer cylinder, and windows are symmetrically disposed on opposite sides of the metal outer cylinder; and liquid level calibration marks are arranged on the transparent inner cylinder corresponding to positions of the windows on the metal outer cylinder.

Preferably, a right end of the transparent inner cylinder is integrally formed with a threaded connector, facilitating the mounting of a syringe rotating cover hand-held connecting rod through the threaded connector; and a one-way valve suction connector is internally housed within a liquid inlet on the threaded connector, used for facilitating connection between the one-way valve suction connector and a liquid storage device through a hose, causing liquid to transfer into the syringe through the one-way valve suction connector.

Preferably, an outer side wall of a head piston in the T-shaped piston adjustment rod is abutted against an inner wall of the syringe cylinder, with a sliding connection between the head piston and the syringe cylinder through mutual engagement.

Preferably, a one-way spring ball valve is housed within the connector, a spring end in the one-way spring ball valve is arranged on an annular cushion block in the connector, and a ball end in the one-way spring ball valve is abutted against a tail end of the hollow rod in the T-shaped piston adjustment rod.

Preferably, the tail end of the hollow rod in the T-shaped piston adjustment rod is arranged as a trapezoidal mouth.

Preferably, elastic clamping blocks are integrally formed on opposite sides of the outer wall of the outer pull cap in a symmetrical manner; and I-shaped clamping holes matched with the clamping blocks through a snap-fit connection are symmetrically disposed on opposite sides of the break-proof buffer cap.

Preferably, an annular rubber gasket is adhered to a central hole of a left end in the break-proof buffer cap in a mutually matched manner, with an inner hole of the annular rubber gasket used for a passage of the steel needle.

The present disclosure has the following beneficial effects. In the present disclosure, the semi-automatic adjustable-dose syringe can prolong the service life of the syringe by changing and adjusting structures and component combinations of the syringe itself, which effectively reduces wear and waste of components, ensures safe operation and fully demonstrates more reasonable ergonomic design methodology. At the same time, the immunization for household and free-range poultry and livestock is more time-efficient and labor-saving, significantly minimizing physical harm and energy consumption of epidemic prevention personnel during immunization of large and medium-sized free-range or penned livestock.

### BRIEF DESCRIPTION OF THE DRAWINGS

To more clearly illustrate the technical solutions in the embodiment of the present disclosure or in the related art, a brief description of the drawings required to be used in the embodiment is presented below. Obviously, the drawings described below are only some embodiments of the present disclosure. For those ordinary skilled in the art, other drawings may be obtained without creative efforts.

FIG. 1 is a schematic structural diagram of a semi-automatic adjustable-dose syringe provided by an embodiment of the present disclosure;

FIG. 2 is a partial enlargement view of portion I in FIG. 1;

FIG. 3 is a schematic structural diagram of an outer pull cap;

FIG. 4 is a schematic structural diagram of a T-shaped piston adjustment rod;

FIG. 5 is a schematic structural diagram of a syringe cylinder; and

FIG. 6 is a cross-sectional schematic diagram of the syringe cylinder.

### REFERENCE NUMERALS AND DENOTATIONS THEREOF

1—syringe cylinder; 2—adjustment cylinder; 3—piston sleeve rod; 4—T-shaped piston adjustment rod; 5—first spring; 6—outer pull cap; 7—second spring; and 8—needle break-proof buffer cap.

### DETAILED DESCRIPTION

The technical solutions in the embodiment of the present disclosure are further described clearly and completely below in combination with the accompanying drawings. Obviously, the embodiment described is only some, rather than all embodiments of the present disclosure. Based on the embodiment of the present disclosure, all other embodiments obtained by those ordinary skilled in the art without creative efforts fall within the scope of protection of the present disclosure.

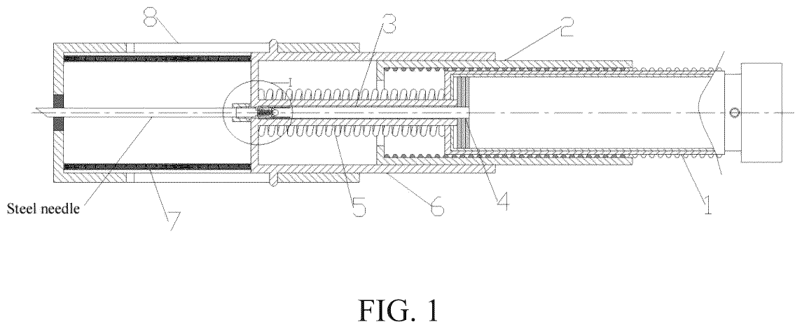

Embodiment 1: referring to FIGS. 1-6, a semi-automatic adjustable-dose syringe includes a syringe cylinder 1, an adjustment cylinder 2, a piston sleeve rod 3, a T-shaped piston adjustment rod 4, a first spring 5, an outer pull cap 6, second springs 7 and a needle break-proof buffer cap 8, and an exterior of a left side of the syringe cylinder 1 is threadedly connected to the adjustment cylinder 2 through mutual engagement; the adjustment cylinder 2 is internally housed within the outer pull cap 6, with a sliding connection between an outer wall of the adjustment cylinder 2 and an inner wall of the outer pull cap 6; the piston sleeve rod 3 is internally housed within the outer pull cap 6, one end of the piston sleeve rod 3 is clamped into a clamping groove in the outer pull cap 6 through mutual engagement, and the other end of the piston sleeve rod 3 penetrates through a through hole at a left end of the adjustment cylinder 2 and extends into the syringe cylinder 1, in which it is to be noted that, after the outer pull cap is assembled with the piston sleeve rod, an inner surface of the outer pull cap can perfectly fit against an outer surface of the hollow rod in the T-shaped piston adjustment rod, while an outer surface of the clamping groove is seamlessly connected to an outer surface of the sleeve rod with smooth surface continuity; and the needle break-proof buffer cap 8 is sleeved on a left end of the outer pull cap 6, and an outer side face of the left end of the outer pull cap 6 is movably connected to an inner side face of a left end of the needle break-proof buffer cap 8 through the second springs 7.

In a further description, the first spring 5 is sleeved on an outer side of the clamping groove inside the outer pull cap 6, one end of the first spring 5 is adhered to the left end of the interior of the outer pull cap 6, and the other end of the first spring 5 penetrates through the through hole on the adjustment cylinder 5 and is adhered to an outer side face of a left end of the syringe cylinder 1, used for facilitating a reciprocating linear movement of the piston, thereby achieving both liquid injection and intake functions for the syringe cylinder 1; a connector matched with a mounting cap in a steel needle through a snap-fit connection is arranged at the left end of the outer pull cap 6; and the T-shaped piston adjustment rod 4 is internally housed within the syringe cylinder 1, and a hollow rod in the T-shaped piston adjustment rod 4 is inserted into the piston sleeve rod and is threadedly connected to internal threads on an inner side of the connector through mutual engagement. It is to be noted that a contact position between the connector and the outer pull cap 6 is closely fitted.

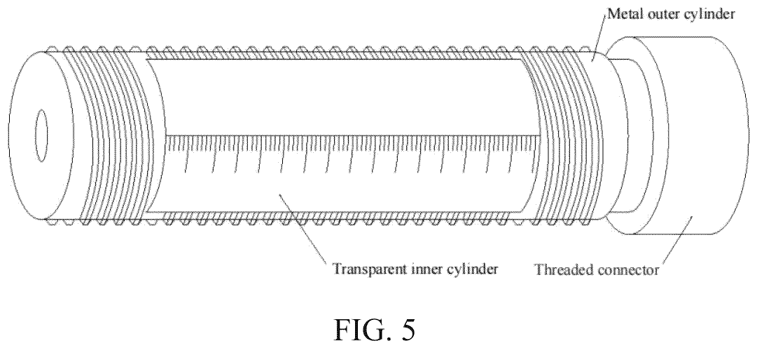

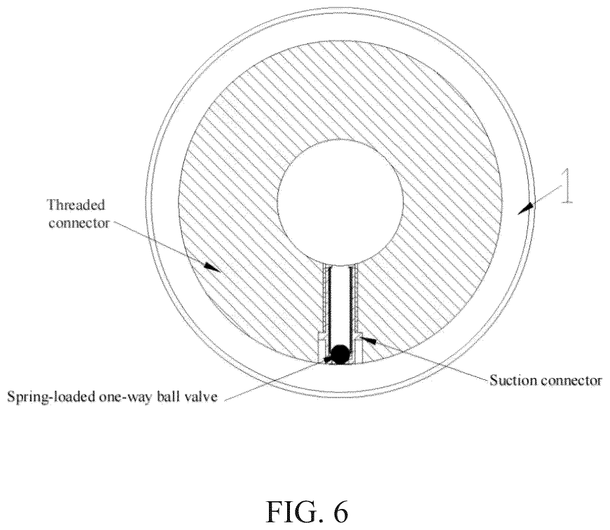

In a further description, in the embodiment, referring to FIG. 1, FIG. 5 and FIG. 6, the syringe cylinder 1 is formed by a transparent inner cylinder and a metal outer cylinder from inside to outside, with the metal outer cylinder sleeved on the transparent inner cylinder; external threads matched with inner threads in the adjustment cylinder 2 are disposed on the metal outer cylinder, and windows are symmetrically disposed on opposite sides of the metal outer cylinder, used for easy observation of calibration marks on the transparent inner cylinder and a liquid level; and liquid level calibration marks are arranged on the transparent inner cylinder corresponding to positions of the windows on the metal outer cylinder.

In a further description, referring to FIG. 1 and FIG. 5, a right end of the transparent inner cylinder is integrally formed with a threaded connector, facilitating the mounting of a syringe rotating cover hand-held connecting rod through the threaded connector. Referring to FIG. 1 and FIG. 6, a one-way valve suction connector is internally housed within a liquid inlet on the threaded connector, used for facilitating connection between the one-way valve suction connector and a liquid storage device through a hose, causing liquid to transfer into the syringe through the one-way valve suction connector. Referring to FIG. 6, the liquid inlet on the threaded connector is threadedly connected to a tail portion of a suction connector in the one-way valve suction connector, and a spring-loaded one-way ball valve in the one-way valve suction connector is arranged inside the suction connector, with the other end of a spring on the spring-loaded one-way ball valve being snap-fitted into a spring clamping groove on the threaded connector.

In a further description, referring to FIG. 1, an outer side wall of a head piston in the T-shaped piston adjustment rod 4 is abutted against an inner wall of the syringe cylinder 1, with a sliding connection between the head piston and the syringe cylinder 1 through mutual engagement.

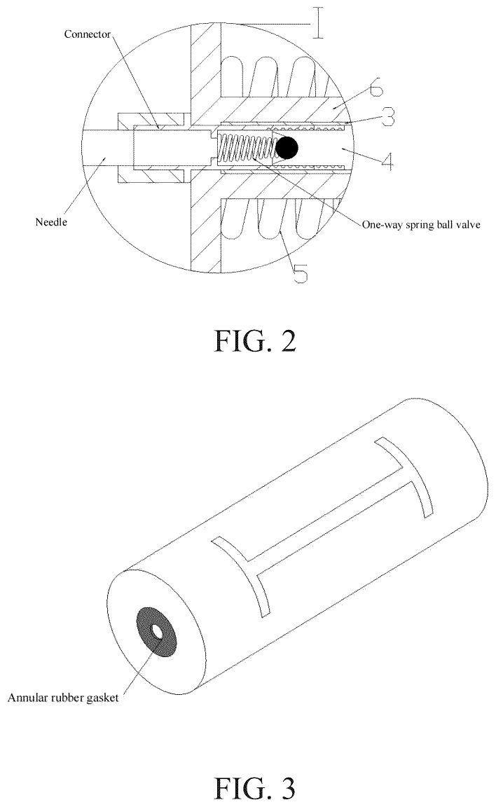

In a further description, referring to FIGS. 1-2, the one-way spring ball valve is housed within the connector, a spring end in the one-way spring ball valve is arranged on an annular cushion block in the connector, and a ball end in the one-way spring ball valve is abutted against a tail end of the hollow rod in the T-shaped piston adjustment rod, in which it is to be noted that, referring to FIGS. 1-2, in the one-way spring ball valve, a diameter of a connection end from the spring to the annular gasket is greater than that of a connection end from the spring to the ball.

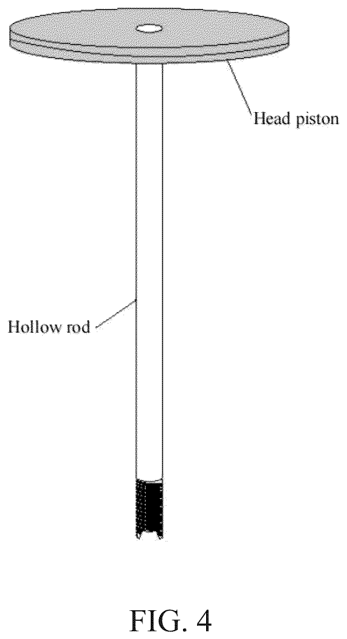

In a further description, referring to FIG. 1 and FIG. 4, the tail end of the hollow rod in the T-shaped piston adjustment rod 4 is arranged as a trapezoidal mouth.

In a further description, referring to FIG. 1, elastic clamping blocks are integrally formed on opposite sides of the outer wall of the outer pull cap 6 in a symmetrical manner. Referring to FIG. 1 and FIG. 3, I-shaped clamping holes matched with the clamping blocks through a snap-fit connection are symmetrically disposed on opposite sides of the needle break-proof buffer cap 8. The elastic clamping blocks (i.e., an elastic latching mechanism) are clamped with the I-shaped clamping holes through mutual engagement, facilitating the disassembly and assembly of the needle break-proof buffer cap, further realizing the quick disassembly and assembly of commercially available steel needles currently on the market.

In a further description, referring to FIG. 1 and FIG. 3, an annular rubber gasket is adhered to a central hole of a left end of the needle break-proof buffer cap 8 in a mutually matched manner, with an inner hole of the annular rubber gasket used for a passage of the steel needle.

During installation, after the syringe cylinder is mounted, the metal outer cylinder of the mounted syringe cylinder is threadedly connected to the adjustment cylinder. After the first spring is fixed inside the outer pull cap, the adjustment cylinder is placed into the outer pull cap to compress the syringe cylinder. At this time, the transparent inner cylinder in the syringe cylinder is adhesively secured to the other end of the first spring, causing the hollow rod of the T-shaped piston adjustment rod to align with the central through hole of the piston sleeve rod in the outer pull cap and place into the syringe cylinder. At this time, the hollow rod of the T-shaped piston adjustment rod is inserted into the piston sleeve rod, and the head piston of the T-shaped piston adjustment rod is placed into the transparent inner cylinder. The one-way spring ball valve is placed into the connector, and the connector is inserted into the other end of the piston sleeve rod and is rotated, causing the connector and the piston sleeve rod to be secured through the rotation of threads. At this time, the connector and the hollow rod of the T-shaped piston adjustment rod are fastened by threaded connection. After the two second springs are symmetrically adhered to the outer side face of the left end of the outer pull cap, the steel needle is mounted on the connector, causing the needle break-proof buffer cap to be sleeved on the outer pull cap with slight compression. At this time, the needle break-proof buffer cap is adhesively secured to the other ends of the second springs, completing the assembly of the syringe in the embodiment. It is to be noted that during installation, the I-shaped clamping holes on the needle break-proof buffer cap need to be aligned with the clamping blocks on the outer wall of the outer pull cap.

In the present disclosure, the semi-automatic adjustable-dose syringe has the following characteristics.

I. An integrated sleeve structure of the syringe can effectively prevent bending, fracturing, or breaking of the steel needle. It achieves initial medication liquid aspiration through manual operation, followed by completing the first injection by a forward push of the hand and arm towards the target. During the retraction of the hand and arm, this syringe automatically aspirates liquid for the next injection (that is, multiple injections can be achieved without repeated aspiration through the elastic force of the spring).

II. Compared to existing gun-type continuous syringes where prolonged single-handed operation of the grip easily causes hand muscle fatigue, this syringe only requires grasping a threaded hand-held connecting rod connected to an exterior of the syringe, significantly alleviating hand fatigue.

III. When injected into medium to large-sized animals, the need of extensive personnel for restraint is eliminated through this syringe, which effectively prevents needle bending, breakage and wear. In contrast, the existing gun-type continuous injectors require multiple personnel and fail to restrain the animals fully, while being unable to prevent needle bending, breakage and wear.

IV. This syringe can quickly, efficiently and safely complete an injection process of epidemic prevention and treatment for free-range and captive animals. The existing gun-type continuous injector exhibits an extremely slow injection speed and a low efficiency, failing to ensure the safety of epidemic prevention personnel, and leading to injuries of varying severity to different body parts.

V. After the injection, the buffer cap rotates to cover the needle, causing the needle to be clamped into the elastic latching mechanism. Moreover, a dose adjustment cylinder rotates, which effectively prevents waste caused by medication leakage, syringe damage and injury of personnel by accidental touch, while the current gun-type injectors lack both needle buffer mechanisms and anti-leakage protection, which can easily cause injury by accidental touch of epidemic prevention personnel, as well as waste and loss of medication (or vaccines).

VI. After being introduced into the market: (1) this syringe can significantly enhance immunization rates for poultry and livestock, effectively control epidemic transmission, and substantially reduce occupational infection risks for breeding practitioners due to physical injuries; (2) when integrated with existing intelligent robotics technologies, this syringe can achieve highly automated breeding treatment and epidemic prevention, thereby improving operational efficiency in animal husbandry and enhancing the health and quality of life of practitioners; (3) the novel, unique, and innovative design of this semi-automatic adjustable-dose syringe will inevitably gain strong recognition from domestic and international manufacturers and consumers. This syringe will occupy a dominant position in the animal vaccination injector market and maintain pricing power over the long term, while leading consumption trends and concepts.

Obviously, for those skilled in the art, a number of improvements and modifications can be made without departing from the spirit and scope of the present disclosure. In this way, if these improvements and modifications of the present disclosure belong to the scope of the claims and equivalents thereof, the present disclosure is also intended to include such improvements and modifications.

1. A semi-automatic adjustable-dose syringe, comprising a syringe cylinder, an adjustment cylinder, a piston sleeve rod, a T-shaped piston adjustment rod, a first spring, an outer pull cap, second springs and a needle break-proof buffer cap, wherein an exterior of a left side of the syringe cylinder is threadedly connected to the adjustment cylinder through mutual engagement; the adjustment cylinder is internally housed within the outer pull cap, with a sliding connection between an outer wall of the adjustment cylinder and an inner wall of the outer pull cap; the piston sleeve rod is internally housed within the outer pull cap, one end of the piston sleeve rod is clamped into a clamping groove in the outer pull cap, and the other end of the piston sleeve rod penetrates through a through hole at a left end of the adjustment cylinder and extends into the syringe cylinder; the needle break-proof buffer cap is sleeved on a left end of the outer pull cap, and an outer side face of the left end of the outer pull cap is movably connected to an inner side face of a left end of the needle break-proof buffer cap through the second springs; the first spring is sleeved on an outer side of the clamping groove inside the outer pull cap, one end of the first spring is adhered to the left end of the interior of the outer pull cap, and the other end of the first spring penetrates through the through hole on the adjustment cylinder and is adhered to an outer side face of a left end of the syringe cylinder; a connector matched with a mounting cap in a steel needle through a snap-fit connection is arranged at the left end of the outer pull cap; and the T-shaped piston adjustment rod is internally housed within the syringe cylinder, and a hollow rod in the T-shaped piston adjustment rod is inserted into the piston sleeve rod and is threadedly connected to internal threads on an inner side of the connector through mutual engagement.

2. The semi-automatic adjustable-dose syringe according to claim 1, wherein the syringe cylinder is formed by a transparent inner cylinder and a metal outer cylinder from inside to outside, with the metal outer cylinder sleeved on the transparent inner cylinder; external threads matched with inner threads in the adjustment cylinder are disposed on the metal outer cylinder, and windows are symmetrically disposed on opposite sides of the metal outer cylinder; and liquid level calibration marks are arranged on the transparent inner cylinder corresponding to positions of the windows on the metal outer cylinder.

3. The semi-automatic adjustable-dose syringe according to claim 2, wherein a right end of the transparent inner cylinder is integrally formed with a threaded connector, facilitating the mounting of a syringe rotating cover hand-held connecting rod through the threaded connector; and a one-way valve suction connector is internally housed within a liquid inlet on the threaded connector.

4. The semi-automatic adjustable-dose syringe according to claim 1, wherein an outer side wall of a head piston in the T-shaped piston adjustment rod is abutted against an inner wall of the syringe cylinder, with a sliding connection between the head piston and the syringe cylinder through mutual engagement.

5. The semi-automatic adjustable-dose syringe according to claim 1, wherein a one-way spring ball valve is housed within the connector, a spring end in the one-way spring ball valve is arranged on an annular cushion block in the connector, and a ball end in the one-way spring ball valve is abutted against a tail end of the hollow rod in the T-shaped piston adjustment rod.

6. The semi-automatic adjustable-dose syringe according to claim 1, wherein the tail end of the hollow rod in the T-shaped piston adjustment rod is arranged as a trapezoidal mouth.

7. The semi-automatic adjustable-dose syringe according to claim 5, wherein the tail end of the hollow rod in the T-shaped piston adjustment rod is arranged as a trapezoidal mouth.

8. The semi-automatic adjustable-dose syringe according to claim 1, wherein elastic clamping blocks are integrally formed on opposite sides of the outer wall of the outer pull cap in a symmetrical manner; and I-shaped clamping holes matched with the clamping blocks through a snap-fit connection are symmetrically disposed on opposite sides of the break-proof buffer cap.

9. The semi-automatic adjustable-dose syringe according to claim 1, wherein an annular rubber gasket is adhered to a central hole of a left end in the break-proof buffer cap in a mutually matched manner, with an inner hole of the annular rubber gasket used for a passage of the steel needle.
